(19) United States
(12) Patent Application Publication
(10) Pub. No.: US 2014/0276095 A1
(43) Pub. Date: Sep. 18, 2014

(54) SYSTEM AND METHOD FOR ENHANCED GONIOMETRY

(71) Applicants: MIRIAM GRIGGS, Scottsdale, AZ (US); Rob Richardson, Gilbert, AZ (US)

(72) Inventors: MIRIAM GRIGGS, Scottsdale, AZ (US); Rob Richardson, Gilbert, AZ (US)

(21) Appl. No.: 13/838,236

(22) Filed: Mar. 15, 2013

(51) Int. Cl. A61B 5/11; A61B 5/00

(52) U.S. Cl. CPC A61B 5/1121; A61B 5/0077
USPC 600/476; 600/595

(57) ABSTRACT

The invention comprises a computer-based system and method for enhanced goniometry using consumer grade data capture components coupled with software to monitor, track, and diagnose joint pain, injuries, and disorders. A video capture device is used to capture and display the movements of a patient in real time and stored in a database for review on a time-delayed basis. The patient movements are digitized by the video capture components and analyzed by the computer software program. By observing variations in joint movements and tracking the variations over time, a medical professional can monitor, evaluate, and diagnose a wide variety of joint diseases, injuries, and disorders. The medical professional can also prepare individualized treatment plans and monitor the results as the patient progresses in treatment and therapy. Additionally, the medical professional may use aggregated anonymous joint movement data to determine “best practices” for future patients, based on past results.

### BACKGROUND OF THE INVENTION

1. Technical Field

The present invention generally relates to movement of the human joints and more specifically relates to the observation and measurement of joint movement.

2. Background Art

Goniometry is the measuring of angles created by the bones of the body at the joints. Typically, medical professionals such as physicians, physical therapists, orthopedic surgeons, sports medicine specialists, occupational therapists, and kinesiologists will use a goniometer to measure joint movement when treating a patient with joint pain or joint damage. A goniometer is an instrument that either measures an angle or allows an object to be rotated to a precise angular position. In physical therapy and occupational therapy, a goniometer is frequently used to measure the axis and range of motion. If a patient or client experiences decreased range of motion in a joint (e.g. a knee or elbow), the physician or physical therapist can use a goniometer to assess what the range of motion is prior to intervention, and then evaluate the efficacy of the intervention by using the goniometer in subsequent clinical evaluations of the patient.

A simple goniometer has a moving arm, a stationary arm, and a fulcrum or body. The fulcrum or body is placed over the joint being measured and on it is a scale with markings ranging from 0° to 180°. The stationary arm will be aligned with the inactive part of the joint measured, while the moving arm is placed on the part of the limb that is moved in the joint's motion. For example, when measuring knee flexion, the stationary arm will be aligned over the thigh in line with the greater trochanter of the femur. The fulcrum is aligned over the knee joint or lateral epicondyle of the femur, and the moving arm with the midline of the leg or lateral malleolus. A trained medical professional can use the goniometry to conduct a series of tests to evaluate the movement of one or more joints of a patient, prior to treatment, during treatment, and after treatment.

Performing these tests and obtaining data on joint movement is important for many reasons. First, observing and measuring the mobility of joints is important for diagnosis and determining the presence or absence of dysfunction. In a chronic condition, goniometry can measure the progression of the disorder. An example of this is the progression of rheumatoid arthritis. Furthermore, joint motion measurement can evaluate improvements or lack of progression during rehabilitation. This not only provides motivation for the patient when there are improvements, but modifications can be made if a given treatment is not effective.

One of the problems with conventional goniometers is the lack of uniformity, precision, and standardization in application. For example, the placement of the goniometer is subjective, at least to some degree. One medical professional will place the device on a patient in one location while another medical professional may select a slightly different location. This may negatively impact the resulting joint movement measurements. Similarly, even with the same medical profession, multiple measurements over an extended period of time will likely be made with the goniometer in slightly different locations, potentially skewing the results and diminishing the certainty of treatment plans and patient progress.

Additionally, the observation of the patient may adversely impact the joint movement measurement process. For example, a malingering patient, in an attempt to remain on disability by simulating or exaggerating pain during an office visit, may feign pain in a joint when, in fact, there is none. However, since the medical professional does not know if the patient is being truthful, the actual improvement of joint strength and mobility may be masked.

Other existing goniometry devices, including photo-goniometers and robotic instruments using physical sensors, are available but suffer from other limitations. These limitations include expense, difficulty in deployment, highly trained operators, etc.

Given the relative uncertainty associated with the current generation of goniometers and the ad hoc application of goniometry in the medical industry today, many medical professionals and their patients may be making important treatment decisions based on less than accurate information. Some doctors, frustrated with the lack of accuracy obtained from traditional goniometers may simply “guess” at the joint measurements. Accordingly, without improvements in the implements and methods used in goniometry, the process and results for many clinical outcomes will continue to be sub-optimal.

### BRIEF SUMMARY OF THE INVENTION

The invention comprises a computer-based system and method for enhanced goniometry using readily available and relatively inexpensive video capture components coupled with software to monitor, track, and diagnose joint pain, injuries, and disorders. At least one video capture device is used to capture the movements of a patient. The patient movements are digitized by the video capture components and analyzed by the computer software program. By observing the variations of the joint movements and tracking the joint movements of the patient over time, a medical professional can monitor, evaluate, and diagnose a wide variety of joint diseases, injuries, and disorders. The medical professional can also prepare individualized treatment plans and monitor the results as the patient progresses in treatment and therapy. Additionally, over time, the medical professional may use aggregated anonymous joint movement data to determine “best practices” for future patients, based on past results.

### BRIEF DESCRIPTION OF THE FIGURES

The preferred embodiments of the present invention will hereinafter be described in conjunction with the appended drawings, wherein like designations denote like elements, and:

FIG. 1 illustrates a prior art goniometer;

FIG. 2 illustrates a system for enhanced goniometry in accordance with a preferred exemplary embodiment of the present invention;

FIG. 3 illustrates visual display for joint measurement using by a system for enhanced goniometry in accordance with a preferred exemplary embodiment of the present invention;

FIG. 4 illustrates visual display for joint measurement using by a system for enhanced goniometry in accordance with a preferred exemplary embodiment of the present invention;

FIG. 5 illustrates a computer used in conjunction with a system for enhanced goniometry in accordance with a preferred exemplary embodiment of the present invention; and

FIG. 6 illustrates a method for enhanced goniometry in accordance with a preferred exemplary embodiment of the present invention.

### DETAILED DESCRIPTION

The invention comprises a computer-based system and method for enhanced goniometry using consumer grade data capture components coupled with software to monitor, track, and diagnose joint pain, injuries, and disorders. A video capture device is used to capture and display the movements of a patient, in real time and on a time-delayed basis. The patient movements are digitized by the video capture components and analyzed by the computer software program. By observing the variations of the joint movements and tracking the joint movements of the patient over time, a medical professional can monitor, evaluate, and diagnose a wide variety of joint diseases, injuries, and disorders. The medical professional can also prepare individualized treatment plans and monitor the results as the patient progresses in treatment and therapy. Additionally, the movement data is stored in a database and over time, the medical professional may use aggregated anonymous joint movement data to determine “best practices” for future patients, based on past results.

Additionally, using one or more preferred embodiments of the present invention it will be possible to measure and establish a “normal” baseline for specific joints and bones for each patient. Since “normal can vary significantly from patient to patient, measurements for each patient will be useful in diagnosis and treatment. For example, using this information, a medical professional will be able to compare a patient's “normal” leg with the other leg to determine what, if any injury or deformity may be present, including determining the specific percentage of movement (e.g., range of motion) that the patient has lost in the injured or deformed limb.

Thus far in physical medicine medical professionals have had great difficulty in coming up with “normal” values for joint (and subsequently muscle) motion. With accurate and reproducible data medical professionals will be able to reduce or eliminate use bias and more easily collect data that will allow them to measure range of motion and normal values for specific populations including different age groups, genders and activity level. This will allow medical professionals to come up with much more specific advice. For instance, at the present time, there is no way to measure complicated angles such as those required to measure and diagnose “posture” related issues. Once the data points for thousands of individual measurements have been captured and analyzed, medical professionals will have the opportunity to develop and issue guidelines about diagnosing normal and abnormal posture positions are based on reproducible data by gender and age. This, in turn, will allow medical professionals to come up with normal values for specific bone and muscle structures such as “normal” hamstring length based on age, sex and activity. A young soccer player, for instance, does not have any specific goals about how to avoid hamstring rupture during play. Pre-sports physicals could easily identify this player if his low back is excessively flat and shortened lumbo-pelvic angle is consistent with very tight hamstrings. This player could be identified out BEFORE injury and allow the medical professional to prescribe specific stretches and goals to achieve to safely participate in the sport and reduce the likelihood of injury.

The preferred embodiments of the present invention will also provide medical professionals with the ability to measure relative “normal” values for bone position and movement on an individual basis. Since these normative values can vary significantly from patient to patient, the medical professional will be able to more effectively evaluate and compare range of motion problems in a given patient. For example, a medical professional will be able to measure range of motion for a patient's “normal” leg with the opposite side injured leg. By comparing the two measurements, the medical professional measure and observe specific percentages of movement degradation due to disease, injury and the like.

Thus far in physical medicine medical professionals have had significant difficulty in coming up with “normal” values for joint and muscle motion. With accurate and reproducible data medical professionals will be equipped to eliminate “use bias” and collect more reliable data that will allow medical professionals to measure range of motion and normal values for specific populations (e.g., different age groups, genders and activity level). This will allow medical professionals to come up with much more specific guidance regarding prevention and treatment of musculoskeletal injuries and disorders. For example, medical professionals presently have no way to measure complicated angles such as those required to measure “posture.” Once medical professionals have been able to capture thousands of individual measurements they will be able to start to make some generalized determinations as to what normal and abnormal postures may be based on reproducible data by gender and age.

This process, in turn, will allow medical professional to establish and promulgate normalized values for certain bone and muscle configurations (e.g., hamstring length or bone length) based on variables such as age, sex and activity. A young soccer player, for instance, does not necessarily have any specific information about how to avoid a hamstring rupture during play. A pre-sports physical could identify physical characteristics of the player and, if the lower back is excessively flat and shortened, the lumbo-pelvic angle would be consistent with very tight hamstrings. The soccer player could be identified prior to any hamstring injury and allow the medical professional or sports trainer to give the soccer player specific stretches and goals to achieve to safely participate in the sport.

Similarly, specific post injury images and related data can be compared to pre-injury images and comparisons can be made in real time without any experience on the part of the medical professional conducting the examination. This will provide for enhanced treatment plans that are more likely to resolve bone and joint issues in a more rapid and repeatable fashion.

Referring now to FIG. 1, a prior art goniometer 100 is depicted. As shown in FIG. 1, goniometer 100 has a first arm 110 and a second arm 120. Arms 110 and 120 are connected at a radial connection 130 and are configured to rotate about pivot point 135. Arm 110 has a series of graduated indicia or markings 111 use in positioning goniometer 100 and measuring joint angles. Arm 120 has a series of graduated indicia or markings 121 use in positioning goniometer 100 and measuring joint angles. Radial connection 130 has a series of graduated indicia or markings 131 use in positioning goniometer 100 and measuring joint angles.

In use, the medical professional will place arm 110 in line with the approximate position of a bone in the patient's body. The medical professional will place arm 120 in line with the approximate position of a second bone in the patient's body. Once arms 110 and 120 are properly positioned, the medical professional can determine the approximate angle between the bones by reading the indicia on radial connection 130. As shown in FIG. 1, arms 110 and 120 may be re-positioned relative to each other to define an angle “A” and an angle “B” as well as another other angles that may need to be measured.

While goniometer 100, and similar prior art goniometers, have been in use for many years, the limitations of these devices are well known to those skilled in the art. Even though approximate angles of joint movement can be measured, the accuracy, efficiency, and consistency of the measurement process is generally considered less than optimal. It is not uncommon for results to be skewed from measurement to measurement, due to human error.

Referring now to FIG. 2, a computer-based system 200 for enhanced goniometry is depicted. As shown in FIG. 2, system 200 comprises a computer 220, an optional consumer grade video interface unit 230 and one or more data capture devices (e.g., a video camera) 240.

In the most preferred embodiments of the present invention, computer 220 may be any type of computer system known to those skilled in the art that is capable of being configured for use with network computer system 200 as described herein. It should be noted that no specific operating system or hardware platform is excluded and it is anticipated that many different hardware and software platforms may be configured to create computer 220. It should be noted that in the most preferred embodiments of the present invention, desktop computer 220 is linked (via wired or wireless connection) to its own LAN or WAN and has access to one or more additional data servers (not shown this FIG.).

Video interface unit 230 and video capture device are standard consumer grade products that are readily available for purchase at retail locations and via the Internet. One preferred embodiment of the present invention is implemented by using an Xbox® coupled with a Kinect® video capture device. In this combination, video capture device 240 (a Kinect® or Leap® Motion® controller) is configured to monitor and digitize a person's movement and transmit the digitized movement data stream to video interface unit 230 and/or to at least one microprocessor contained in or associated with computer 220. Those skilled in the art will recognize that the inclusion of multiple video capture devices 240 will tend to provide more detailed images and enhance data integrity for creating bone and joint visualizations.

Computer 220 is equipped with a specialized user interface and computer program that presents the digitized movements of patient 250 on the screen of computer 220 for viewing by a user of computer 220 in real time via a graphical user interface. It is important to note that the use of video interface unit 230 is optional since in at least one preferred embodiment of the present invention the functions of video interface unit 230 are included in the software used by computer 220. In this embodiment, no external video interface unit 230 is required. It should also be noted that video capture devices captures a data stream that may include audio as well as video signals for processing by computer 220. Additionally, in some preferred embodiments of the present invention, video interface unit 230 may provide additional functionality for certain applications. For example, may provide a series of on-screen movements displayed for the patient to observe and mimic in real time. As the patient mimics the movements displayed on screen (e.g., a golf swing, jumping jacks, etc.) the patient's movement will be recorded and analyzed in real time (or stored for later review and analysis) to detect any anomalies. This will help the medical professional in the diagnosis and treatment of any bone or joint defects or injuries.

An optional printer and an optional fax machine (not shown this FIG.) may also be deployed for various hard copy data output requirements and may be considered to be any standard peripheral devices used for transmitting or outputting paper-based documents, notes, transaction details, reports, etc. in conjunction with the various requests and transactions processed by network computer system 200 (e.g., reports, statistical analyses, automated letters, etc.). Finally, it should be noted that the optional printer and the optional fax machine are merely representative of the many types of peripherals that may be utilized in conjunction with network computer system 200. It is anticipated that other similar peripheral devices will be deployed in the various preferred embodiment of the present invention and no such device is excluded by its omission in FIG. 2.

At least some preferred embodiments of computer system 200 comprise a mobile communication device 290. Mobile communication device 290 is representative of any type of portable cellular device or telephone that may be communicatively coupled to network computer system 200. This includes, for example, personal digital assistants (“PDAs”), Windows® mobile phone devices, Android® OS devices, Palm® OS devices, Pocket PC® devices, the Apple® iOS® devices and other various types of smartphones. Those skilled in the art will recognize that these various devices and others are suitable for deployment as mobile communication device 290. While somewhat less powerful than computer 220, mobile communication device 290 may also be configured to wirelessly communicate with computer 220 to send and retrieve tracking and messaging services related information to and from computer 220.

Given the standard functionality for devices that may be deployed as mobile communication device 290, this communication be provided by a wireless Internet connection (e.g. “wi-fi” or “wi-max”) or a Bluetooth® connection. One example of the use for mobile communication device 290 in the context of network computer system 200 would to send messages or alerts to a patient or medical professional regarding the movements and measurements obtained by computer system 200. For example, a doctor could receive mobile messages via mobile communication device 290 from computer system 200 to alert the doctor about the progress of a patient who movements are being monitored by computer system 200.

In the most preferred embodiments of the present invention, at least a portion of network 215 comprises a standard wired or wireless connection between at least some of the components of network computer system 100 for providing access to additional network resources and other remote locations. Network 215 provides for communication between the various components of network computer system 100 and allows for relevant information to be transmitted from device to device. In this fashion, a user of network computer system 100 can quickly and easily gain access to the relevant data and information utilized to search, retrieve, and display information from one or more databases as described in conjunction with the preferred embodiments of the present invention. The data stream may be transmitted in real time or on a time delay basis from a patient's home to a distant medical professional or medical facility to provide for diagnosis at a later time or the data stream may be used in real time for evaluations conducted during appointments in the medical professionals office.

In the most preferred embodiments of the present invention, network 215 is configured to provide relatively high-speed transmission of both audio and video data and signals (and, in some embodiments, environmental and ambient conditions) and comprises at least an Internet connection for transmission of data captured by one or more computers 170 or 180 for transmission of an audio signal to and from a standard phone connection. The phone connection may be interfaced to a standard phone system typically found in most homes and commercial facilities, including for example, the existing “land line” phone system infrastructure and/or digital cellular phone communication systems.

In addition to the other components shown in FIG. 2, a wireless communication access device 275 may be communicatively coupled to network 215 and may be type any wireless communication mechanism that is known to those skilled in the art to provide for wireless communication between network 215 and the various devices associated with network 215, including computer 220 as well as mobile communication device 290. The most preferred embodiments of an acceptable wireless communication access device may comprise any type of wireless bridge, wireless router, or wi-fi “hotspot.” Regardless of the specific components, physical nature, and topology, network 215 serves to logically and communicatively link the physical components of network computer system 200, thereby enabling stable and consistent communication between the components.

In the most preferred embodiment of the present invention, system 200 is used to assist medical professionals in monitoring, diagnosing, and treatment of joint related pain, injury, and disorder for one or more patients. In operation, the components of system 200 cooperate to capture and digitize the movements of patient 250, then transfer a data stream containing the digital images that have been captured to computer 220. After storing the data stream in a database and processing the data stream, a screen associated with computer 220 will display a patient's joint movement on a screen. This image may include a digital representation of all bones and/or joints, some bones and/or joints, and may or may not include an image of the patient extracted from the data stream. Based on the application, the medical professional will selectively display or hide each of these elements. Additionally, system 200 can display the angle between any pair of bones and the distance between multiple joint locations for each patient as the data is extracted from the data stream.

In the most preferred embodiments of the present invention, video capture device 240 will track the movements of patient 250 and create a data stream comprising a series of patient movements that provide point data for determining the position of joints and bones in patient 250. The data stream is sent from video capture device 240 to computer 220 and then through network 215 to a server where it is stored in a local or cloud-based database, and retrieved from the server by another computer, tablet, or mobile device across the Internet. The data contained in the data stream is accumulated over time, providing a historical trend and data for tracking the progress or lack of progress as a patient receives treatment. Additionally, the spatial relationship between the joints and the bones of each patient are typically unique enough (similar to a fingerprint) so as to enable system 200 to identify a patient by the position and relationship of the joints and bones. The skeletal tracking function captures the 3-dimensional (e.g., x, y, and z) coordinates or each joint and the 2-dimensional (e.g., x and y) coordinates for each joint in real time. This data is stored in database 323 of FIG. 3 and is processed by goniometry mechanism 327 of FIG. 3 to calculate joint articulation and rotation positions for creating a skeletal overlay displayed by system 200. Additionally, the location of the joints and bones relative to the limbs, torso and head of the patient can be calculated or extrapolated by system 200 so as to overlay the digital bone and joint locations on a digital image of the patient as captured by data capture device 240.

As the data stream for each patient is captured and stored in database 323 of FIG. 3, the accumulated data will enable faster measurement of the bone and joint positions, more repeatable and reliable data on bone and joint positions, remove observer bias from the determination of the bone and joint positions, and more accurately detect fraud and malingering for making and evaluating insurance and disability decisions.

System 200 will extrapolate and calculate the joint articulation and rotation positions relative position of the patient's bones from the digital images captured by video capture device 240. Using the data extracted from the digital images, system 200 generates a plurality of joint and bone positions 251 and displays the joint and bone positions as well as joint articulation and rotation positions on a screen for review. Bone positions 251 are displayed as lines that represent a digitized version of patient 250 depicting the locations of the major bones in patient 250, as provided by video capture device 240. Each bone position 251 corresponds to a bone in the body of patient 250 and the intersection of two bone positions lines 251 indicates the location of a joint (e.g., knee joint, elbow joint, etc.). The operator of system 200 can interact with a graphical user interface to specify which bone positions 251 are relevant for the diagnosis and treatment of a specific patient 250 and selectively “hide” bone positions 251 that are not needed.

A user interface will allow the medical professional to select one or more bone positions 251 and receive visual feedback such as a display of the angle between the bones represented by bone positions 251 and a histogram or chart showing the relative improvement or lack of improvement over time for the selected bones represented by bone positions 251. This information is all stored in a local or remotely accessible database for retrieval and analysis. In the most preferred embodiments of the present invention, the medical professional will calibrate or tweak the algorithms used to identify the bone and joint locations in the patient to align with the specific patient.

For example, the default algorithm used to identify the location of a bone or joint in a patient may initially locate the outer surface of a patient's arm or leg and basically “split the difference” to determine the location of the bone inside the arm. However, for some patients, this default positioning algorithm may not accurately reflect the patient's bone structure. In that case, the medical professional can specify an offset in any or all of the x, y, and z coordinates to more accurately reflect the bone positioning for a given patient. In this fashion, it is possible to customize the bone positioning to create a more accurate diagnosis and treatment plan.

Additionally, since system 200 can capture and record the entire position and movement of all major bones and joints, it will be possible to record and review data regarding the patient's posture. Over time, with bone position data collected and aggregated from a plurality of patients, the correct bone positions for healthy posture can be ascertained. This will allow a medical professional to more quickly and efficiently diagnose posture related problems, providing the potential for preventative measures to be taken prior to permanent damage occurring in the patients musculoskeletal system.

Similarly, athletic performance may be tracked and compared to baseline performance as a way to measure improvement. Further, mirror image joints (e.g., left side of the body knee joint and right side of the body knee joint) can be compared to each other to determine if a joint disorder is bi-lateral or isolated to a single joint. System 200 is also capable of monitoring bone and joint position in three-dimensional space, allowing for the tracking of movement in all three directions (e.g., x, y, and z plane coordinates).

In addition to the use of bone positions 251, certain preferred embodiments of the present invention may include additional input sensors to enhance the ability of the medical professional to diagnose and treat pain. For example, a thermometer, and/or a heart monitor or pulse oximeter can be affixed to an appropriate location on the patient and used to provide additional data points to further enhance the diagnosis as the patient performs various joint movements. Coupled with the data points from the bone positions 251, the medical professional will have access to a more complete dataset regarding the patient's physical condition.

Similarly, the speed of movement and outliers (e.g., minimum and maximum bone and joint movement parameters) can also be used to aid the medical professional in diagnosing and treating bone and joint diseases and disorders. For example, if a patient correctly produces the prescribed moves, but later reaches out to put on their shoes, the system can highlight this disparity of motion, and correctly identify the fraud. Similarly, if a patient is asked to repeat a particular motion—especially quickly—a patient with a true injury will stop at the same position every time while a malingering patient's motion will vary.

Other applications include the use of an outpatient exercise trainer or therapy program (e.g., a game that prescribes certain movements for the patient to mimic) that can be used to document the date and time and motion of each exercise performed by a patient. This data can be uploaded to the medical professional's office, immediately or on a time-delayed basis. This will let the medical professional know if the patient has been following the prescribed therapy program. Similarly, the system can infer patient height and size from the bone and joint data in the captured data stream, and infer if the subject performing the exercises is indeed the patient who was seen in the office visit. For example if the patient in the office was 5′4 and the subject performing the exercises is 6′2, then compliance with the prescribed treatment or therapy program has not been met.

Referring now to FIG. 3, computer 220 of FIG. 2 suitably comprises at least one Central Processing Unit (CPU) or processor 310, an auxiliary storage interface 340, a display interface 345, and a network interface 350, all of which are interconnected via a system bus 360. Note that various modifications, additions, or deletions may be made to computer 220 illustrated in FIG. 3 within the scope of the present invention such as the addition of cache memory or other peripheral devices. FIG. 3 is not intended to be exhaustive, but is presented to simply illustrate some of the more salient features of computer 220. Those skilled in the art will recognize additional variations that may be useful in certain applications.

Processor 310 performs computation and control functions of computer 220, and most preferably comprises a suitable central processing unit (CPU). Processor 310 may comprise a single integrated circuit, such as a microprocessor, or may comprise any suitable number of integrated circuit devices and/or circuit boards working in cooperation to accomplish the functions of a processor or CPU. Processor 310 is configured to execute one or more software programs contained within main memory 320. Although computer 220 of FIG. 3 contains only a single main processor 310 and a single system bus 360, it should be understood that the present invention applies equally to computer systems having multiple processors and multiple system buses. Similarly, although system bus 360 of the preferred embodiment is a typical hardwired, multi-drop bus, any connection means that supports bi-directional communication in a computer-related environment could be used.

Auxiliary storage interface 340 allows computer 220 to store and retrieve information from auxiliary storage devices, such as external storage mechanism 370, magnetic disk drives (e.g., hard disks or floppy diskettes) or optical storage devices (e.g., CD-ROM). One suitable storage device is a direct access storage device (DASD) 380. As shown in FIG. 3, DASD 380 may be a DVD or CD-ROM drive that may read programs and data from a DVD or CD disk 390.

Display interface 345 is used to directly connect one or more displays 375 to computer 220. Display 375, which may be non-intelligent (e.g., “dumb”) terminals or fully programmable workstations, are used to provide system administrators and users the ability to communicate with computer 220. Additionally, in certain preferred embodiments of the present invention, computer 220 may have an integrated display 375.

Network interface 350 is used to connect computer 220 to network 215 and computer 220 of FIG. 1. Network interface 350 broadly represents any suitable way to interconnect electronic devices, regardless of whether the network comprises present day analog and/or digital techniques or via some networking mechanism of the future. Network interface 350 preferably includes a combination of hardware and software that allows communications on network 215.

Software provided in conjunction network interface 350 preferably includes a communication manager that manages communication with other computer systems or other network devices via network 215 using a suitable network protocol. Many different network protocols can be used to implement a network. These protocols are specialized computer programs that allow computers to communicate across a network. TCP/IP (Transmission Control Protocol/Internet Protocol) is just one example of a suitable network protocol that may be used by the communication manager contained within network interface 350.

It is important to note that while the present invention has been (and will continue to be) described in the context of a fully functional computer system with certain application software, those skilled in the art will appreciate that the various software mechanisms of the present invention are capable of being distributed as a program product in conjunction with an article of manufacture comprising software stored on a computer readable storage medium in a variety of forms, and that the various preferred embodiments of the present invention applies equally regardless of the particular type or storage medium used to actually carry out the distribution. Examples of computer readable storage media include: non-transitory recordable type media such as DVD and CD ROMS disks (e.g., disk 390), and transmission type media such as digital and analog communication links, including wireless communication links.

Main memory 320 suitably contains an operating system 321, a web browser 322, one or more databases 323, a user interface 324, a communication server 325, a security mechanism 226, and a goniometry mechanism 327. The term “memory” as used herein refers to any storage location in the virtual memory space of computer 220. Those skilled in the art will recognize the actual size and location of memory 320 is not a critical part of data storage and may possible implementations may be selected, based on the application environment.

It should be understood that main memory 220 might not necessarily contain all parts of all components shown. For example, portions of operating system 321 may be loaded into an instruction cache (not shown) for processor 310 to execute, while other files may well be stored on magnetic or optical disk storage devices (not shown). In addition, although database 323 is shown to reside in the same memory location as operating system 321, it is to be understood that main memory 320 may consist of multiple disparate memory locations. It should also be noted that any and all of the individual software mechanisms or components shown in main memory 320 might be combined in various forms and distributed as a stand-alone program product. Finally, it should be noted that additional software components, not shown in this figure, might also be included.

Operating system 321 includes the software that is used to operate and control computer 220. In general, processor 310 typically executes operating system 321. Operating system 321 may be a single program or, alternatively, a collection of multiple programs that act in concert to perform the functions of an operating system. Any operating system now known to those skilled in the art or later developed may be considered for inclusion with the various preferred embodiments of the present invention.

Web browser 322 may be any web browser application currently known or later developed for communicating with web clients over a network such as the Internet. Examples of suitable web browsers 322 include Microsoft Internet Explorer®, Safari®, Chrome®, Firefox®, and the like. Additionally, other vendors have developed or will develop web browsers that will be suitable for use with the various preferred embodiments of the present invention. Regardless of the specific form of implementation, Web browser 322 provides access, including a user interface, to allow individuals and entities to interact with the Internet and graphical user interface 324, including via network 215 of FIG. 1. The Internet connectivity described herein provides for “cloud-based” storage of data, including the ability to transmit the data from the cloud to multiple disparate locations.

Database 323 is representative of any suitable database known to those skilled in the art. In the most preferred embodiments of the present invention, database 323 is a Structured Query Language (SQL) compatible database file capable of storing information relative to various items that may be of interest to the users of computer 220 of FIG. 2. In the most preferred embodiments of the present invention, database 323 will comprise a plurality of information that may be useful to an organization or individual that wants to perform joint movement monitoring, as well as preparing and implementing treatment plans and programs, in conjunction with a preferred embodiment of computer 220 of FIG. 2. This will most often include patient data, diagnosis and treatment data for each patient, historical patient information for electronic medical records (“EMR”), patient treatment plans, etc. As previously mentioned, the data collected may be stored in the “cloud” and accessed via the Internet. Or, computer 220 of FIG. 2 may be deployed as a locally accessed computer with limited connectivity.

Graphical user interface is a software component that provides the users of computer 220 of FIG. 2 a means for interacting with the various components of computer 220. In the most preferred embodiments of the present invention, the operator of computer 220 will be able to see a graphical representation of the patient and the patient's movements displayed on a monitor. The operator will be able to see the movements of the patient and the movements of the corresponding bones and joints that are depicted by graphical user interface 324. The operator can use a mouse or other input device to “point and click” on bones and joints of interest to highlight or focus on the desired joints and bones. Additionally, the operator can display multiple images of the patient that have been captured over time and, by superimposing the images, view a visual representation of the patient's progress or regression over a user-specified period of time.

In at least one preferred embodiment of the present invention adapted for tracking, monitoring and recording joint movement in a medical environment, database 323 will include a plurality of database records containing information about multiple patients and doctors as well as information about treatment plans and programs as well as information providing for tracking, analyzing and reporting joint movement and measurement data that may be used to provide various services to the prospective users of computer 220 of FIG. 2. By storing patient related data for a plurality of patients in database 323, the data can be used in a variety of ways. For example, the bone and joint movement data for a large group of patients can be aggregated and “anonymized” for use in research and studies. By aggregating data relative to certain demographic groups and certain bone diseases and disorders, enhanced diagnostic and treatment programs can be created and medical professional will be able to tap into a resource that does not exist with prior art solutions.

Those skilled in the art will recognize that other types of information for other types of data that may be used in other applications (e.g., historical, informational, technical, etc.) may be stored and retrieved as well. While database 323 is shown to be residing in main memory 320, it should be noted that database 323 might also be physically stored in a location other than main memory 320. For example, database 323 may be stored on external storage device 370 or DASD 380 and coupled to computer 220 via auxiliary storage I/F 340. Additionally, while shown as a single database 323, those skilled in the art will recognize the database 323 may actually comprise a series of related databases, logically linked together. Depending on the specific application and design parameters, database 323 may take many different forms when implemented.

The most preferred embodiments of computer 220 of FIG. 2 will typically include a communication server 325 in main memory 320. Communication server 325 is a programmable system that is capable of generating one or more forms of automated or “on demand” messages or message events. For example, communication server 325 may be configured to send automated email messages or SMS text messages to cell phones. Communication server 325 may also be used to generate hard copy messages (e.g., mail merge letters) that are then sent via standard U.S. Postal Service or some type of commercial message delivery company.

Additionally, communication server 325 may be configured to generate a facsimile message by utilizing fax server and a facsimile modem (not shown this FIG.) that is contained in computer 220 of FIG. 2. Communication server 325 is also capable of being configured and used to send and receive various electronic status messages (e.g. audio and video alerts) and updates to computer 220 of FIG. 1, as may be necessary to enhance the overall process of completing activities related to the provision of monitoring, tracking, and calculating bone and joint positioning data as described herein.

This includes the generation of automated messages, including emails and audio files, relating to the tracking and reporting of joint movement as well as prediction and response services as well as sending informational messages related to medical personal, patients, insurance companies, employers, etc. Automated or on-demand e-mail messages may also be generated to provide notifications regarding the status of a patient's progress as well as other information for related to various treatment programs that may be implemented by an medical professional in response to the treatment of joint pain, injury, and disorder in accordance with the various preferred embodiments of the present invention. This will allow a medical professional to review and analyze patient information remotely and in a “time shifted” manner.

It is anticipated that communication server 325 will be configured to generate and transmit audio message events that contain a plurality of audio files or “clips” where each audio message event may contain multiple discrete elements. For example, each audio message event may contain standardized pre-recorded audio clips; audio clips generated by an automated text-to-speech computer program (e.g., from a medial professional's chart of the patient), and contemporaneously recorded audio clips that are unique to a specific audio message event. Additionally, dynamic audio clips, using elements extracted from database 323, that are associated with a patient interview or treatment session recorded during an office visit with a specific patient, may also be included in the audio message events. For example, a generic introduction of the medical professional associated with a specific patient may be included, a facility identifier (e.g., hospital or medical facility associated with the patient), the name of the patient, as well as the purpose for transmission of the audio message event (e.g., second opinion, analysis for insurance claim, etc.). The audio message event may be transmitted to multiple persons, including a supervisory physician, insurance company representative, or the patient as well.

In addition, most preferred embodiments of the present invention would include a security and/or encryption mechanism 326 for verifying access to the data and information contained in and transmitted to and from computer 320. Security mechanism 326 may be incorporated into operating system 321 and/or web browser 322. Additionally, security mechanism 326 may also provide encryption capabilities for other components of computer 220 of FIG. 2, thereby enhancing the robustness of computer 220 of FIG. 2. Security mechanism 326 is most preferably configured to protect the integrity and security of the information transmitted via network 215 of FIG. 2.

Further, depending on the type and quantity of information stored in database 323 and accessed by graphical user interface 324, security mechanism 326 may provide different levels of security and/or encryption for different computer 220 of FIG. 2 and the information stored in database 323. The level and type of security measures applied by security mechanism 326 may be determined by the identity of the end-user and/or the nature of a given request and/or response. In some preferred embodiments of the present invention, security mechanism 326 may be contained in or implemented in conjunction with certain hardware components (not shown this FIG.) such as hardware-based firewalls, switches, dongles, and the like. When working with protected health information (“PHI”), it is important to protect and secure the patient's data and this capability will be provided by security mechanism 326 so as to remain in compliance with various laws and statutes (e.g., Health Insurance Portability and Accountability Act “HIPAA”).

Goniometry mechanism 327 is a software program or mechanism that uses the information stored in database(s) 323 to capture and track bone and joint movement for one or more patients for use in providing medical evaluations and treatments for bone and joint pain, injuries and disorders. Goniometry mechanism 327 will access the data contained in database(s) 323 and perform various calculations related to joint movement, including changes in range of motion, outliers, joint positioning, speed of bone and joint movement, etc.

In the most preferred embodiments of the present invention, goniometry mechanism 327 comprises one or more algorithms that use a series of captured joint positions to identify joint related issues and assist medical professionals in creating and monitoring the effectiveness of various treatment plans and programs. In at least one preferred embodiment of the present invention, the functions of video interface unit 230 of FIG. 2 are included in goniometry mechanism 327. For many applications, goniometry mechanism 327 will interpret the data provided by system 200 to provide a visualization of the bone and joint location, based on a pre-configured algorithm.

By locating the edges of a patient's body (e.g., the surface points of the arms, legs, neck, head, etc.), the algorithm will extrapolate the location of the bones beneath the skin. Using the intersection of the bones as a starting point, the algorithm will also extrapolate and calculate or determine such information as bone length and joint location. By accessing a database of standardized measurements for bone lengths based on physical attributes (e.g., age, sex, weight, etc.) then the algorithm will be able to fine-tune the bone and joint positioning data. The bone and joint location information is then transformed into a digital image of the skeletal structure of the patient and the skeletal structure can be displayed on a computer monitor or screen. Additionally, the actual photographic image of the patient can be simultaneously displayed, with the skeletal structure displayed as an overlay. The skeletal structure information can be captured in real time and reviewed at any time. The medical professional can use this information as the basis for diagnosis and treatment.

Referring now to FIG. 4, a screen 422 associated with computer 220 of FIG. 2 displays a first joint position visualization. As shown in FIG. 3, a pair of bone positions 251, representing two bones in subject 250 from FIG. 2, form an angle 410. Angle 410 may represent, for example, the relative positions of the two large bones of the leg (e.g., the femur and tibia bones with the intersection representing the knee joint). Angle 410 may be a measurement of bone positions prior to providing treatment for patient 250. As previously explained, bone positions 251 are created from the video input of patient 250 as captured by video input device 240.

Referring now to FIG. 5, a screen 422 associated with computer 220 of FIG. 2 displays a second joint position visualization. As shown in FIG. 5, a pair of bone positions 251, representing two bones in subject 250 from FIG. 2, form an angle 510. Angle 510 may be a measurement of bone position after providing treatment to patient 250. As previously explained, bone positions 251 are created from the video input of patient 250 as captured by video input device 240. By comparing the magnitude of angle 410 with the magnitude of angle 510, the increase or decrease in the range of mobility for the joint represented by pair of bone positions 251 can be measured. This will allow the medical professional to determine whether or not the patient is making progress, based on a standardized measurement. The locations of joints can also be determined and represented on a screen as well. The improved process for capturing the bone and joint data will provide for a more effective and efficient data collection process. This, in turn, provides for significantly enhanced repeatability and confidence in the results of the treatment program because the medical professional has more reliable and accurate data.

As shown in FIG. 2, FIG. 4, and FIG. 5, joint articulation and rotation positions, including the joint positions and angles between various bones in the body of a patient 250, can be calculated using the various bone positions 251 as calculated by system 200. Each bone position 251 represents the positions of joints and bone in the body of patient 250. By digitally observing and recording the visual images captured by movement of patient 250 over time, it will be easier for medical professionals to diagnose and treat bone injuries and disorders. Additionally, since the accuracy and reliability of the measurement of bone movement and joint angles will be significantly enhanced, it will be easier to detect “malingering” by employees seeking disability pay or compensation. Typically, malingering patients will have difficulty accurately reproducing the same joint motion when required to perform the motion repeatedly or at varied speeds. Accordingly, by capturing and comparing multiple attempts at predetermined joint movements, gathered during the same or from previous visits, subterfuge can be more easily detected by the medical professional. As part of the process, the actual angles between bones can be measured and displayed on the screen, thereby providing visual and numerical data points for the medical professional to use in diagnosis and treatment of bone and joint disorders and injuries.

Referring now to FIG. 6, a method 600 for enhanced goniometry in accordance with a preferred exemplary embodiment of the present intention is depicted. As shown in FIG. 6, the first step is generally to capture bone and joint data for the patient (step 610). This includes patient identifying information, symptoms, etc. This also includes the gathering of joint and bone positioning and movement data. The joint and bone data can be automatically captured by positioning the patient in front of a video capture device, such as video capture device 240 of FIG. 2. The patient joint data, as captured, can be viewed in real time and will be processed and stored in a database (step 620) for later analysis and reporting. Part of processing the data will typically include generating a real time display of the patient bone and joint data, including joint articulation and rotation positions, on a screen for analysis by a medical professional in a clinical setting.

As shown in FIG. 6, as long as there is a need or desire to capture more data (step 630=“YES”), the system will continue to capture data. This is an iterative process that can be started and stopped as desired and/or necessary. In a clinical setting, the data capture process is conducted in real time and may include movement modifications suggested by a medical professional on an ad hoc basis or as part of a structured movement session. For example, a “game” where the patient is instructed to make various movements in front of the video capture device could be utilized.

Once enough data has been captured (step 630=“NO”), then the patient bone and joint data can be reviewed and analyzed (step 640) and any anomalies or concerns can be identified (step 650). It is important to note that the “trigger” for an anomaly can be a preset default reading such as the relative or absolute position of one bone relative to another, or a user-identified trigger (e.g., pain associated with a specific joint or bone movement or position) that is selected and set on a patient-by-patient basis. Once an anomaly has been identified (step 650=“YES”), then the medical professional can analyze the specific patient joint data and provide a diagnosis and, if necessary, implement or update a treatment plan (step 660) to address the specific needs of the patient.

The review of the data stream for the patient's movements may be viewed in real time in a medical professional's office or it may be “time” and “place” shifted for more convenience or expediency. For example, a small town may not have a large enough patient population to justify a full time orthopedic doctor on staff at a local clinic. However, by using the various preferred embodiments of the present invention, a patient can schedule an appointment at a small clinic where a data stream containing their bone and joint movements can be captured. The data stream can be transmitted to a distant location where a qualified medical professional can view the data stream, observe the bone and joint movement and positioning data, and provide diagnostic and treatment options without ever having met the patient. This will provide the opportunity for many more people to receive high quality medical care where it would not otherwise be available. Similarly, the medical professional can monitor the patient over time to update the diagnosis and treatment plan as desired. This separates the place and time restrictions that are so prevalent in the profession today.

It should be noted that the treatment plan may be devised by the medical professional based on the personal experience of the medical professional or it may be automatically generated based on an aggregated dataset of similar anomalies that have been gathered from multiple patients over an extended period of time. This will be especially helpful for situations where the medial professional is inexperienced or for situations where a trained medical professional is not available. For example, it is anticipated that the various preferred embodiments of the present invention can be used in countries where medical professionals with joint disorder experience are not available. By accessing the aggregated dataset, generic treatment plans can be recommended, based on the patient joint profile generated by the system.

With the treatment plan in place or updated, the medical professional can continue to monitor the progress of the patient (step 670) directly or remotely and store or communicate the results (step 680) as desired or necessary. For example, in a distance medicine environment, the joint data and treatment progress may be transmitted to a medical professional or medical facility in a distant geographical location via the Internet or other communication media where the data will be analyzed by a trained medical professional. Security mechanism 326 of FIG. 3 can be used to encrypt the data stream as necessary to protect relevant personal medical data during transmission. This may be important for compliance with various laws, statutes and regulations government the storage and transmission of sensitive data such as medical records.

In this environment, a patient ID and related data capture date can be transmitted to a medical professional or medical facility for diagnosis. The treatment plan can then be updated as necessary or desired. This process will allow patients without ready access to a medical professional to receive high quality care over a distance. In some instances, the data may suggest that a patient needs to come into a medical facility for an in-person evaluation, based on the data sent to the medical professional or medical facility. It would be possible to establish “trigger points” in the patient's bone and joint movement data that would automatically generate a message or notification to the medical professional. Similarly, if a patient was not performing the prescribed exercises or movements prescribed by the medical professional, the system could generate a report to the medical professional to let them know that intervention may be necessary.

Additionally, after use over time, the database will eventually contain a significant amount of data from multiple patients over an extended period of time. The results from various treatment plans for different patients can be de-personalized, stored and aggregated and normalized to determine the best treatment protocols for various joint injuries and disorders. The data can be categorized by various demographic factors (e.g., sex, age, physical condition, etc.) so as to parse the dataset and extract the most relevant data by matching the search parameters to match a specific patient's needs. This data can be used as a predictive methodology as well as a reactive methodology to diagnose joint issues and create or update treatment plans. With all this accomplished, the process can be repeated as frequently and for as long as necessary or desired to achieve the goals or objectives of the treatment plan.

In yet another preferred embodiment of the present invention, standardized movements can be stored in the database and used to train people on the mechanics of proper lifting. By superimposing the actual movement of a person over the idealized movement for lifting, deviations from the correct form can be noted. This may be useful for occupational testing when employees are requited to lift objects using “appropriate mechanics.” This will allow a physical therapist to evaluate the patient's functional movement. Additionally, the system may be used by the medical professional to monitor and capture the patient's movements during an entire office visit, including times when it may not be obvious that the patient's movements are being monitored. This will reduce or eliminate the issues associated with observational impacts and provide educational opportunities. For example, proper posture while sitting at a desk could be modeled, or the proper stance for placing items on tall shelves could be demonstrated. By observing and mimicking the correct posture, the patient can learn how to avoid injury and/or reduce the pain associated with everyday movements. Eventually, the patient's monitored movements can be aligned with the idealized movements.

Further, the system as described herein may be useful in disability evaluation environments. One problem with disability testing is detecting whether or not a patient is experiencing actual pain or is consciously or unconsciously exaggerating the presence and magnitude of their pain. In some circumstances, Waddell testing may be used to more accurately gauge the level of pain or discomfort a patient is experiencing. As the patient's movements are captured by the system, it will provide a more accurate and repeatable measurement for establishing a baseline as well as changes to the pain profile associated with joint movements. This will enhance compliance and ensure that patients are treated properly.

From the foregoing description, it should be appreciated that a system and method for apparatus and methods for enhanced goniometry is provided by the various preferred embodiments of the present invention and that the various preferred embodiments offer significant benefits that would be apparent to one skilled in the art. The various embodiments described herein are useful for a wide variety of applications including physical therapy diagnosis and treatment, patient compliance with treatment plans, patient training and assistance, sports training, anonymized statistical analysis, etc.

Furthermore, while multiple preferred embodiments have been presented in the foregoing description, it should be appreciated that a vast number of variations in the preferred embodiments exist. Lastly, it should be appreciated that these embodiments are preferred exemplary embodiments only and are not intended to limit the scope, applicability, or configuration of the invention in any way.

Rather, the foregoing detailed description provides those skilled in the art with a convenient road map for implementing a preferred exemplary embodiment of the invention, it being understood that various changes may be made in the function and arrangement of elements described in the exemplary preferred embodiment without departing from the spirit and scope of the invention as set forth in the appended claims.

1. An apparatus comprising:
at least one processor;
a memory coupled to the at least one processor;
a data capture device, the data capture device capturing a data stream, the data stream comprising a plurality of bone and joint data; and
a goniometry mechanism residing in the memory, the goniometry mechanism being configured to:
extract the bone and joint data from the data stream;
calculate joint articulation and rotation from the bone and joint data; and
display at least some of the bone and joint positions on a screen.

2. The apparatus of claim 1 wherein the data stream in stored in a database for later review and analysis.

3. The apparatus of claim 1 wherein the data stream is displayed on the screen in real time and then stored in a database.

4. The apparatus of claim 1 wherein the data capture device comprises a plurality of data capture devices and wherein each of the plurality of data capture devices captures a data stream and wherein the goniometry mechanism is configured to analyze an aggregated data stream comprising each of the data streams captured by the plurality of capture devices.

5. The apparatus of claim 1 further comprising a security mechanism residing in the memory, the security mechanism encrypting at least a portion of the data stream prior to displaying the at least some of the bone and joint positions on the screen.

6. The apparatus of claim 1 further comprising a user interface residing in the memory, the user interface creating an image on the screen, the image comprising:
an image of a patient;
a skeletal structure derived from the bone and joint positions superimposed on the image of the patient; and
a plurality of bone and joint measurements including at least one bone length and at least one angle measurement between a first bone and a second bone.

7. The apparatus of claim 1 wherein the goniometry mechanism comprises a user adjustable positioning algorithm and wherein the bone and joint positions extracted from the bone and joint data by the goniometry mechanism can be adjusted by the user.

8. The apparatus of claim 1 wherein the goniometry mechanism is configured to:
extract the bone and joint data from a first data stream at a first point in time;
calculate a first set of joint articulation and rotation positions from the bone and joint data at the first point in time;
extract the bone and joint data from a second data stream at a second point in time;
calculate a second set of joint articulation and rotation positions from the bone and joint data at the second point in time; and
simultaneously display at least some of the first set of joint articulation and rotation positions from the first point in time and the second set of joint articulation and rotation positions from the second point in time on the screen.

9. The apparatus of claim 1 further comprising a web browser residing in the memory, the web browser being configured to interact with a user and the goniometry mechanism to provide access to the database and the bone and joint data from the data stream.

10. The apparatus of claim 1 further comprising a communication server residing in the memory, the communication server being configured to transmit at least one message related to the bone and joint data from the data stream.

11. A method comprising the steps of:
using a video capture device to capture bone and joint data for at least one patient in a data stream;
transferring the data stream to a computer;
extracting the bone and joint data from the data stream;
calculating joint articulation and rotation data from the bone and joint data; and
displaying at least some of the joint articulation and rotation data on a screen.

12. The method of claim 11 further comprising the step of analyzing the bone and joint data, including joint articulation and rotation data, to identify any anomalies.

13. The method of claim 11 wherein the step of using a video capture device to capture at least one patient's bone and joint data in a data stream comprises the step of capturing a plurality of bone and joint data for a plurality of patients and further comprising the steps of:
aggregating and anonymizing the data to create at least one joint articulation and rotation database; and
accessing the at least one baseline joint articulation and rotation database to perform diagnostic analysis and create treatment plans for at least one bone or joint disorder or disease.

14. The method of claim 11 further comprising at least one of the following steps:
displaying an image of the at least one patient on a computer screen;
accessing a user interface to superimpose a portion of the joint articulation and rotation data on the image of the at least one patient; and
accessing a user interface to remove a portion of the joint articulation and rotation data that had been previously superimposed on the image of the at least one patient.

15. The method of claim 11 further wherein the steps of using a video capture device to capture bone and joint data for at least one patient in a data stream and displaying at least a portion of the joint articulation and rotation data on a screen occur in other than real time.

16. The method of claim 11 further comprising the step of displaying a plurality of angles and position information regarding the joint articulation and rotation data for the at least one patient.

17. The method of claim 11 further comprising the steps of:
monitoring the data stream for the at least one patient over time to create a database of historical patient data; and
updating a treatment plan for the at least one patient based on the historical patient data.

18. The method of claim 11 further comprising the steps of:
collecting a plurality of data streams from a plurality of patients;
aggregating and averaging the plurality of data streams for a specific group of patients to create a normalized bone and joint structure dataset;
storing the normalized bone and joint structure dataset in a database; and
comparing a data stream from a single patient to the normalized bone and joint structure dataset to determine a diagnosis or treatment plan for the single patient.

19. The method of claim 11 further comprising the steps of:
transmitting the bone and joint data from the database to a mobile communication device; and
displaying a patient image and a skeletal image on a visual display associated with the mobile communication device.
